(19) United States
(12) Patent Application Publication
(10) Pub. No.: US 2023/0221299 A1
(43) Pub. Date: Jul. 13, 2023

(54) A DEVICE FOR STUDYING INTERACTIONS OF A FIRST CELL TYPE WITH A SECOND CELL TYPE AND RELATED METHOD

(71) Applicants: Agency for Science, Technology and Research, Singapore (SG); National University of Singapore, Singapore (SG)

(72) Inventors: Christopher Peter Tostado, Singapore (SG); Yi-Chin Toh, Queensland (AU); Ramanuj Dasgupta, Singapore (SG)

(21) Appl. No.: 17/996,593

(22) PCT Filed: Apr. 22, 2021
(86) PCT No.: PCT/SG2021/050227

§ 371 Date: Oct. 19, 2022

(30) Foreign Application Priority Data
Apr. 22, 2020 (SG) 10202003690W

(51) Int. Cl. G01N 33/50; B01L 3/00

(52) U.S. Cl. CPC G01N 33/5011; B01L 3/502746; B01L 2300/0819; B01L 2300/0867; B01L 2300/0874; B01L 2400/086; B01L 2400/0409

(57) ABSTRACT

There is provided a microfluidic device comprising a first region configured to hold target cells, e.g., tumor cells, a second region configured to hold effector cells, e.g., immune cells, and an array of microstructures disposed between the first and second regions, wherein the first region is in fluid communication with the second region, and wherein the array of microstructures is configured to selectively allow movement of immune cells, from the second region to an interaction zone that is at least partially disposed within the first region, for interaction with tumor cells in the interaction zone. The array of microstructures can be an array of micropillars. Also provided is a chip comprising a plurality of the device and a method of studying interactions of a first cell type with a second cell type.

### TECHNICAL FIELD

The present disclosure relates broadly to a device and a method for studying interactions of a first cell type with a second cell type.

### BACKGROUND

Cancer is a leading cause of death and expensive to treat. The health care costs to society associated with treating cancer, which affects older adults most often, will only increase in the future considering humans are now living longer. Today, medical treatments, even for cancer, are often not focused on a personalized patient care approach. Where feasible, a more personalized approach to treating cancer can lead to potentially more accurate prediction of how a therapy will affect an individual. Better predictions of how individual patients will respond to treatment benefits not only health care providers, but can also lower costs for insurers and give patients a better peace of mind by knowing that therapies are individually prescribed.

For a certain class of blood-derived cancers such as acute lymphoblastic leukemia (ALL), lymphomas and multiple myelomas, the cutting-edge of cancer treatment lies in a relatively new therapy called adopted cell transfer (ACT). This “living” treatment uses the patient's own immune cells, modifies them, and reintroduces them into the body, using the patient's own immune system to destroy the cancer. These immune-cell mediated therapies can be combined with previously established chemotherapeutic standard-of-care drugs or combinations thereof, to form completely new types of cancer therapies. There is now an opportunity to apply these immunotherapy breakthroughs not only to haematological cancers but to solid tumor cancers. However, there remain both scientific and practical challenges to doing this, namely the difficulty in predicting how immune cells will interact with solid tumor bodies as well as the high cost, risks, and patient-sensitive nature of this type of research. Therefore, it is increasingly difficult to conduct studies in this area and progress in developing new types of cancer treatments is slowed.

In view of the above, there is a need to address at least ameliorate the above-mentioned problems. In particular, there is a need to provide a device and method for studying interactions between different cells (e.g., immune cells and tumor cells) that address or at least ameliorate the above-mentioned problems.

### SUMMARY

In one aspect, there is provided a microfluidic device comprising a first region configured to hold tumor cells; a second region configured to hold immune cells; and an array of microstructures disposed between the first and second regions, wherein the first region is in fluid communication with the second region, and wherein the array of microstructures is configured to selectively allow movement of immune cells, from the second region to an interaction zone that is at least partially disposed within the first region, for interaction with tumor cells in the interaction zone.

In one embodiment, the first and second regions are symmetrical about a same line of symmetry.

In one embodiment, the device further comprises one or more third regions, the third region being in fluid communication with the first and second region, wherein the array of microstructures comprises microstructures disposed between the third region and the first region.

In one embodiment, the array of microstructures comprises microstructures disposed between the third region and the second region.

In one embodiment, the third region substantially surrounds the first region.

In one embodiment, the first, second and third regions are symmetrical about a same line of symmetry.

In one embodiment, the array of microstructures comprises microstructures organised in a radial manner or a grid-like manner.

In one embodiment, the array of microstructures comprises microstructures organised as substantially concentric rows of microstructures.

In one embodiment, the distance between the microstructures in the row closest to the first region is smaller than the distance between the microstructures in the row furthest from the first region.

herein the size of the microstructures in the row closest to the first region is smaller than the size of the microstructures in the row furthest from the first region.

In one embodiment, each of said regions comprises a shape defined by tapering of a bigger area to a smaller area.

In one embodiment, the device further comprises ports corresponding to each of said regions for providing access to each of the regions.

In one embodiment, the device comprises a seeding layer and a support layer, wherein the ports are disposed on the seeding layer and the corresponding regions are disposed on the support layer.

In one embodiment, the first region has a larger depth than the second region.

In one embodiment, the second region has substantially the same depth as the third region.

In one aspect, there is provided a chip comprising a plurality of the device disclosed herein.

In one aspect, there is provided a method of studying interactions of a first cell type with a second cell type, the method comprising: providing a device comprising a first region configured to hold a first cell type; a second region configured to a second cell type; and an array of microstructures disposed between the first and second regions, wherein the first region is in fluid communication with the second region, and wherein the array of microstructures is configured to selectively allow movement of the second cell type from the second region to an interaction zone that is at least partially disposed within the first region, to allow interaction of the first cell type and the second cell type in the interaction zone; seeding the first cell type in the first region of the device; applying a first external force to direct the first cell type to the interaction zone; seeding the second cell type in the second region of the device; allowing the second cell type to migrate from the second region to the interaction zone for interaction with the first cell type in the interaction zone; and monitoring migration of the second cell type and interaction of the second cell type with the first cell type.

In one embodiment, the method further comprises, subsequent to the monitoring step, applying a second external force to direct cells present within the interaction zone away from the interaction zone for retrieval and analysis.

In one embodiment, the monitoring step comprises monitoring the migration and interaction of the cells with an image capturing apparatus.

In one embodiment, the device further comprises one or more third regions, and the method further comprises, prior to the step of applying the first external force, seeding microenvironment materials into the one or more third regions, wherein the third region is in fluid communication with the first and second regions, and wherein the array of microstructures comprises microstructures disposed between the third region and the first region.

### DEFINITIONS

The term “micro” as used herein is to be interpreted broadly to include dimensions from about 1 micron to about 1000 microns and from about 1 micron to about 100 microns.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals.

Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of a device and a method for studying interactions of a first cell type with a second cell type are disclosed hereinafter.

There is provided a device (e.g., a microfluidic device) comprising a first region, the first region configured to hold at least a first cell type (e.g., target cells such as tumor cells); a second region disposed, the second region configured to hold at least a second cell type (e.g., effector cells such as immune cells); and an array of barriers (for example, an array of structures or an array of microstructures) disposed in a position that is between the first and second regions, wherein the first region is in fluid communication with or fluidly coupled to the second region (e.g., via at least one flow passage/path that is disrupted by the array of barriers), and wherein the array of barriers is configured to selectively allow movement of cells (i.e., the second cell type or immune cells) to an interaction zone (for example, an entrapment area) to allow interaction of the first and second cell types in the interaction zone. The first and second regions may be disposed on a support layer. Advantageously, in various embodiments, the device may provide the capability of compartmentalizing and controlling the interaction between tumor and immune cell populations that the device is intended to examine. Further advantageously, various embodiments of the device, which may be a compartmentalized microfluidic device, may control and direct tumor-immune cell interactions as well as create a complex three-dimensional tumor microenvironment to more closely mimic physiological conditions in-vivo.

Thus, various embodiments of the device may, for example, be used for early discovery or screening of drug/immune therapy combinations, and/or tumor-infiltrating lymphocyte migration studies (e.g., for the study of tumour-immune cell interactions in the presence of chemotherapeutic drugs.). That is, in various embodiments, the device may, for example, be used to yield results of screening for novel combination drug therapies, which may yield potentially effective therapies for cancer treatment (in the early drug discovery stage).

In various embodiments, the microfluidic device comprises a first region configured to hold tumor cells; a second region configured to hold immune cells; and an array of microstructures disposed between the first and second regions, wherein the first region is in fluid communication with the second region, and wherein the array of microstructures is configured to selectively allow movement of immune cells, from the second region to an interaction zone that is at least partially disposed within the first region, for interaction with tumor cells in the interaction zone. In some embodiments, the interaction zone is also at least partially disposed within the second region.

In various embodiments, the selective movement of cells (i.e., the second cell type or immune cells) to the interaction zone comprises selective movement of cells from the second region to the interaction zone. In various embodiments, the interaction zone is an interaction zone of the first region. For example, the interaction zone may in some embodiments be defined as a zone that is completely disposed in the first region. In some other embodiments, the interaction zone may be a zone that spans across the first region and the second region and optionally other regions. Depending on the exact configuration of the array of microstructures and the cell types, the location where interactions between the first and second cell types occur may vary. The selective movement of second cell type may partially arise from the array of barriers (e.g., an array of microstructures) between the first and second regions which present a migratory barrier to the second cell type during the movement from the second region to the interaction zone of the first region. In various embodiments, the array of barriers (e.g., an array of microstructures) are additionally configured to substantially prevent movement of the tumor cells from the first region to the second region e.g., in the presence of an external force such as a rotational or centrifugal force.

In various embodiments, the device is a two layered structure. The device may comprise a seeding layer and a support layer. For example, the device may further comprise a seeding layer disposed over the support layer, the latter having the various regions disposed thereon. The device may comprise ports for providing access to the regions, for example, via ports corresponding to each of said regions for providing access to each of the regions. In various embodiments, the seeding layer comprises a first seeding port that allows access to (e.g., is in fluid communication with, coupled to, or fluidically connected to) the first region for seeding/removal the first cell type in the first region; and a second seeding port that allows access to the second region for seeding/removing the second cell type and/or removal of air bubbles in the second region.

In various embodiments, the device further comprises one or more third regions for one or more of the following functions: seeding/removal of the second cell type, removal of air bubbles and to hold/provide/remove tumor microenvironment (TME) materials. Tumor microenvironment materials may, for example, include artificial extra cellular matrices (e.g., Matrigel), cellular materials (e.g., cancer associated fibroblasts), or both. Tumor microenvironment materials may also include one or more molecular components (e.g., Polysaccharides, Collagen, Glycoproteins, Cytokines), natural materials (e.g., Alginate, Chitosan), and/or cellular/tissue-based components (e.g., lymph vessels, blood vessels, endothelial cells, myeloid derived suppressor cells, macrophages, T-cells and NK-cells). Advantageously, in various embodiments, including TME materials in the device may enhance the physiological relevance of experiments performed on the device (e.g., immune-tumor interaction experiments) by allowing for the creation of a complex three-dimensional TME surrounding the central tumor spheroid being studied.

In various embodiments, the device comprises one or more third regions, for example, at least one, at least two, at least three, at least four or at least five third regions. The device may comprise one or more third region(s) for seeding/removal of the second cell type and/or removal of air bubbles. In one example, the device comprises a single third region for seeding/removal of the second cell type and/or removal of air bubbles. The device may comprise one or more third region(s) configured to hold tumor microenvironment materials. In one example, the device comprises a plurality of third regions (e.g., three third regions), each configured to hold tumor microenvironment materials. In various embodiments, at least one third region (or each of the third region) is in fluid communication with or fluidly coupled to the second region (e.g., via at least one channel/passage/path). Further, in various embodiments, the array of barriers (e.g., the array of microstructures) comprises microstructures disposed between at least one of the third regions and the second region. In some embodiments, the microstructures may be disposed between each of the third regions and the second region. Even further, in various embodiments, at least one of the third region (or each of the third region) is in fluid communication with the first and second regions, wherein the array of barriers (e.g., the array of microstructures) comprises microstructures disposed between at least one third region and the first region and/or between at least one third region and the second region and/or between two different third regions. In some embodiments, the microstructures may be disposed between each of the third regions and the first region, and/or between each of the third regions and the second region. In various embodiments, at least a portion/part of the third region(s) is sandwiched between the first region and the second region. In various embodiments, at least one of the third regions or each of the third regions substantially surrounds the first region. In one embodiment, at least one of the third regions (or a plurality of or all of the third regions) completely surrounds the first region. In other embodiments, where there are two or more separate third regions for example, one of the third regions may completely surround the first region and the other third region (s) may flank or substantially surround the first region. At least part of the third region (or at least part of each of the third region) may be in the interaction zone. In other words, in some embodiments, at least part of the interaction zone may be disposed in one or more third regions. Accordingly, in some embodiments, the interaction zone may span across the first region, the second region and one or more third regions. In various embodiments, the third region(s) are disposed on the support layer.

In various embodiments, the device further comprises a third port (e.g., disposed on said seeding layer) that allows access to (e.g., is in fluid communication with or fluidly coupled to) the third region for one or more of: seeding/removal of the second cell type, removal of air bubbles or to hold/provide/remove tumor microenvironment materials. In various embodiments, the device disclosed herein may allow for retrieval of cells via the various ports described (e.g., the first, second and third seeding ports). The extracted cells may be analysed to yield useful results (e.g., to identify potentially effective therapies for cancer treatment in the early drug discovery stage).

In various embodiments, one or more of the regions or each region (i.e., the first region, second region and third region) may comprise a shape defined by tapering of a bigger area to a smaller area. In one example, the shape may resemble a bulbous shape or a teardrop shape. Further, in various embodiments, the diameter of the curvature of the bigger area of each region may be same, e.g., to allow/accommodate the use of an automatic handler such as a bioprinter to seed/extract cells from the bigger areas.

In various embodiments, one or more regions may each have depth(s) that are deeper than other region(s). In various embodiments, the regions may have different depths from one another in order to accommodate the sizes of the different cells or tumor microenvironment materials being seeded for the particular biological model/application being simulated on the device. In some embodiments, the first region may have a depth that is deeper than the second and/or third region(s). For example, the first region has a depth of no less than about 50 μm to no more than about 500 μm, no less than about 50 μm to no more than about 400 μm, no less than about 50 μm to no more than about 300 μm, no less than about 50 μm to no more than about 290 μm, no less than about 50 μm to no more than about 280 μm, no less than about 50 μm to no more than about 270 μm, no less than about 50 μm to no more than about 260 μm or no less than about 50 μm to no more than about 250 μm. In such examples, the second and/or third region(s) may have a depth of no less than about 15 μm to no more than about 100 μm, no less than about 20 μm to no more than about 90 μm, no less than about 25 μm to no more than about 80 μm, no less than about 25 μm to no more than about 70 μm, no less than about 25 μm to no more than about 60 μm, or no less than about 25 μm to no more than about 50 μm, relative to the top surface of the support layer.

In various embodiments, the second cell type (e.g., immune cells) is capable of moving (or migrating) to the interaction zone only through the spaces between the array of barriers (or array of microstructures).

In various embodiments, the first and second cell types are different cell types. In various embodiments, the different regions/compartments may each contain a single cell population or a coculture of cell types (e.g., fibroblasts and macrophages; cancer-associated fibroblasts (CAFs) and tumour-associated macrophage (TAMs)).

In various embodiments, the first and second cell types may be of different sizes.

In various embodiments, the first cell type is a tumour cell. The first cell type may be derived from a human (e.g., a human sample and/or a patient-derived cell line) or from a non-human mammal/an animal model (e.g., derived from a syngeneic mouse model).

In various embodiments, the first cell type may be co-cultured with one or more other cell types different from the first cell type. The one or more other cell types may be a cell in a tumour microenvironment (TME). For example, the one or more other cell type may be a fibroblast (e.g., a cancer-associated fibroblast (CAF)). In one instance, a tumour microenvironment cell type (e.g., fibroblasts) may be seeded in the first region via the first seeding port (prior to seeding the first cell type e.g., in the form of tumour spheroids) such that the tumour microenvironment cell type may adhere to the spaces in between the array of barriers (e.g., artificial pillar barriers) and form a more physiologically accurate environment for the first cell type e.g., in the form of tumour spheroids.

In various embodiments, the second cell type is an immune cell or a combination of different classes/types of immune cells. For example, the second cell type may be a lymphocyte including T cells such as a CD8+ T cell/a cytotoxic T cell, and a Natural Killer (NK) cell. The second cell type may be derived from a human (e.g., a human sample and/or a patient-derived cell line) or from a non-human mammal/an animal model (e.g., derived from syngeneic mouse model).

In various embodiments, the second cell type may be co-cultured with one or more other cell types different from the second cell type. The one or more other cell types may be a cell in a tumour microenvironment (TME). For example, the one or more other cell type may be one or more of a macrophage (e.g., a tumour-associated macrophage (TAM)), an effector T cell (e.g., a helper T cell/a CD4 T cell, a memory T cell), or a regulatory T cell or a combination thereof.

In various embodiments, the first cell type may be in singularized or spheroid forms. The first cell type may also be in the form of cell aggregates. Accordingly, in various embodiments, the device disclosed herein may be configured to allow loading and compacting of (single) cells into a cellular mass, and/or to trap cellular masses larger than a single cell (e.g., via the array of barriers or microstructures).

In various embodiments, the array of barriers/structures (or array of microstructures) may have an arrangement that resembles a curved shape, a non-polygonal shape and/or a shape that is devoid of sharp corners and/or edges. For example, the arrangement may resemble an overall ellipsoidal shape, circular shape, oval shape or part thereof.

In various embodiments, the array of barriers (or array of microstructures) may be organised in a radial manner, for example in a radiating fashion from a common point of radiation e.g., a common center. That is, in various embodiments, when the array of barriers is organised in a radial manner, the barriers may share the common point of radiation from which the pillars appear to emanate from and the barriers may each be disposed at a distance with respect to the common point of radiation. When organised in a radial manner, the barriers/microstructures may be further arranged in radiating rows, each row being perceptibly distinguished from the other rows. For example, adjacent barriers/microstructures within a row may be separated by a substantially fixed/constant distance from each other and the value of this fixed distance may vary from one row to another such that it is possible to perceptibly make out one row from another. Additionally or alternatively, each row may be sufficiently spaced out/apart from each other such that it is possible to perceptibly identify different groupings of the barriers/microstructures as different rows. The radiating rows may each have a shape that resembles an arc of a full or partial circle such that the microstructures/barriers in each row appears to line the circumference of the arc. Accordingly, in various embodiments, the spaces between adjacent rows also resemble an arc of a full or partial circle.

In various embodiments, the array of barriers may be organised in a manner where the distances between the barriers are uniform, or non-uniform e.g., the array of barriers may be organised in a manner where there is a gradient of distances between the barriers from the outermost row of barriers (furthest from the interaction zone) to the innermost row of the barriers (closest to the interaction zone). For example, when the array of barriers or microstructures are organised in a radial manner, the barriers in the outermost row may be spaced apart at a distance of no less than about 25 μm, no less than about 20 μm, no less than about 15 μm, no less than about 10 μm, no less than about 9 μm, no less than about 8 μm, no less than about 7 μm, no less than about 6 μm or no less than about 5 μm; and the barriers in the innermost row may be spaced apart at a distance of no more than about 15 μm, no more than about 10 μm, no more than about 9 μm, no more than about 8 μm, no more than about 7 μm, no more than about 6 μm or no more than about 5 μm. Consequently, the barriers within the innermost row may be spaced apart at a distance much lesser than the distance in the outermost row; and the barriers within the intermediate rows (between outermost row and innermost row) may be spaced apart at distances falling between those of the outermost row and innermost row e.g., based on graduated increments or decrements. Likewise, the array of barriers may also be organised in a manner where there is a gradient of distances between adjacent rows of barriers (row-to-row distance). For example, the row-to-row distances may change from the outermost rows of barriers (furthest from the interaction zone) to the innermost rows of the barriers (closest to the interaction zone). It will be appreciated that other combinations of non-uniform distances may also be applied.

In various embodiments, the distances between the barriers within a row and/or between different/adjacent rows may be determined based on a size distribution of the second cell type. The distances between the barriers within a row and/or between different/adjacent rows may also be determined based on an attempt to artificially mimic barriers naturally found between tissue types within a body (e.g., an organism) or based on the extent to which a user desires to challenge the ability of the second cell type (e.g., an immune cell) to migrate towards a target (or a first cell type, e.g., a tumour cell). The distances between the barriers within a row and/or between different/adjacent rows may further also be determined based on the intrinsic properties of the second cell type, e.g., size, migration characteristics, deformability. The various embodiments described above may advantageously provide selective permeability at a radial micropillar structure.

In various embodiments, the array of barriers (or array of microstructures) may be organised as substantially concentric rows of microstructures. In various embodiments, the concentric rows are substantially circular in shape. When the array of barriers is organised as substantially concentric rows of microstructures, the microstructures may share a common center and the microstructures may each be disposed at a distance with respect to the common center. In various embodiments, the microstructures/barriers within each row may have substantially the same distance or are substantially equidistant from the common center. In various embodiments, organising the array of barriers as substantially concentric rows of microstructures may be a form or a subset of organising the array of barriers in a radial manner. In various embodiments, when the microstructures are organised in substantially concentric rows (e.g. the rows are substantially circular in shape or part of a circle), the respective/adjacent rows are separated from one another, for example, by a distance of no less than about 10 μm, a distance of no less than about 20 μm, a distance of no less than about 30 μm, a distance of no less than about 40 μm, a distance of no less than about 50 μm, no less than about 60 μm, no less than about 70 μm, no less than about 80 μm, no less than about 90 μm or no less than about 100 μm. The distances between concentric rows may be uniform or non-uniform. In various embodiments, the space created between the concentric rows of microstructures may usefully provide space for the device to hold tumor microenvironment materials in the vicinity of the interaction zone of the first region. Therefore, concentric rows of microstructures may at least partially line the boundaries of the third region (or each of the plurality of third regions), for example to separate a third region from another third region, to separate a third region from the first region, and/or to separate a third region from the second region such that the second cell type has to migrate/move across the respective rows of microstructures when transiting from one region to another. In various embodiments, the concentric rows of microstructures may be further arranged such that the distance between the microstructures in the row closest to the first region is smaller than the distance between the microstructures in the row furthest from the first region, i.e., the distances between the microstructures are non-uniform across different rows (although the distances between the microstructures within the same row may be uniform). Further, in various embodiments, the size or dimensions of the microstructures in the row closest to the first region may be smaller than the size or the dimension of the microstructures in the row furthest from the first region. In various embodiments, size may be defined as the amount of estate space occupied on the support layer and may directly be correlated to the volume, cross-sectional area etc. of the microstructures.

In various embodiments, the array of barriers (or array of microstructures) may be organised in a grid-like manner. For example, the spaces existing between the barriers/microstructures appear to look like a network of substantially perpendicular lines intersecting one another. The array of barriers may be further organised in a manner where the distances between the barriers are uniform. For example, the barriers may be spaced apart at a distance of no more than about 5 μm, no more than about 10 μm, no more than about 15 μm, no more than about 20 μm or no more than about 25 μm. It will be appreciated that other distances may also be applied. In various embodiments, the distances between the barriers may be determined based on a size distribution of the second cell type. The distances between the barriers may also be determined based on an attempt to artificially mimic barriers naturally found between tissue types within a body (e.g., an organism) or based on the extent to which a user desires to challenge the ability of the second cell type (e.g., an immune cell) to migrate towards a target (or a first cell type, e.g., a tumour cell). The distances between the barriers may further also be determined based on the intrinsic properties of the second cell type, e.g., size, migration characteristics, deformability.

The organisation of the array of microstructures may be a combination of two or more of the different organisational patterns described above.

In various embodiments, the array of barriers (or array of microstructures) comprises pillars. In other words, the device may comprise micropillars. For example, the pillars have a non-polygonal cross-section (e.g., circular, oval, elliptic, oblong). Alternatively, the pillars may also have a polygonal cross-section, such as a parallelogram like a square, rectangle or rhombus. In various embodiments, the height of the pillars is the same as the height of the support layer of the device. Thus, depending on the depth of region at which the pillars are disposed (e.g., when they line the boundaries of the regions), the heights of the pillars may vary accordingly. The pillars may have a height of no less than about 50 μm to no more than about 500 μm, no less than about 50μm to no more than about 400 μm, no less than about 50 μm to no more than about 300 μm, no less than about 50 μm to no more than about 290 μm, no less than about 50 μm to no more than about 280 μm, no less than about 50 μm to no more than about 270 μm, no less than about 50 μm to no more than about 260 μm, no less than about 50 μm to no more than about 250 μm; no less than about 15 μm to no more than about 100 μm, no less than 20 μm to no more than about 90 μm, no less than 25 μm to no more than about 80 μm, no less than 25 μm to no more than about 70 μm, no less than 25 μm to no more than about 60 μm, no less than 25 μm to no more than about 50 μm, or no less than about 15 μm and no more than about 100 μm. In various embodiments, the pillars lining the boundaries of the first region have heights that are greater than the pillars lining the boundaries of a third region and/or second region.

In various embodiments, the device may further comprise one or more obstacles configured to or arranged to direct fluid flow in a passive manner. In one example, an obstacle may be arranged between the second region and the array of barriers (or array of microstructures). The obstacle may be arranged to follow the shape of the array of barriers at its circumference (e.g., a curved shape, a non-polygonal shape and/or a shape that is devoid of sharp corners and/or edges). In various embodiments, barriers which direct fluid flow may also be present at other places within the device.

In various embodiments, the device is symmetrical about a plane (or a central axis) that cuts across the centre of the support layer and/or seeding layer.

In various embodiments, the first and second regions are symmetrical about a same/common line of symmetry. In various embodiments, the first region, second region and third region (or each of the third regions) are symmetrical about a same/common line of symmetry. In various embodiments, the first region, second region, and/or third region (or each of the third regions) has/have only a single line of symmetry.

In various embodiments, the device may be manufactured using soft lithography. To improve the resolution of the microstructures within the device as well as the depth of each region, in various embodiments, the manufacturing method may alternatively be converted to an injection molding microfabrication method.

An exemplary device is described below for illustrative purposes. In one example, the device may be a microfluidic device that allows for the observation of cellular interactions, which has the components of: (a) a fluidic chamber partitioned by a radial micropillar barrier into multiple compartments, with said barrier exhibiting a gradient of distances between micropillars and (b) a loading and cell retrieval mechanism which utilizes centrifugal force to seed and recover cells of interest. In the above exemplary embodiments, the microfluidic device may comprise micropillars that act as a trap for the tumor cell mass, and additionally present a barrier to the free movement of lymphocytes. Also, the device may be configured to allow cells to be loaded into the micropillar trap and retrieved from the device via centrifugal forces.

There is also provided a chip (or plate) device comprising a plurality of the devices (i.e., microfluidic devices) provided herein. For example, the chip may be a multiplexed chip comprising a plurality of the devices arranged in an A×B array, where A and B are integers that are independently selected from 1 to 50. Advantageously, in various embodiments, the device design (i.e., the microfluidic device sign) may be scaled to conform to standard industrial dimensions (e.g., the size of a standard microscope size or a well plate). That is, the various embodiments of the device disclosed herein may provide a design with built-in amenability to scale-up using at least two types of multiplexed devices in the form of a chip or plate. Therefore, in various embodiments, the chip (or plate) device may enable massively parallel in vitro screens of immune-tumour interactions. These screens can be conducted with a library of immunotherapy drug candidates to determine which patients respond well to those drugs.

In various embodiments, the chip is be designed and dimensioned to fit conventional centrifugation machines such that a longitudinal axis or a line of symmetry of each of the device (or the regions within the device) contained within the chip approximately passes through the axis of rotation of the centrifugation machines (to allow the centrifugal force to direct the cells contained therein to one end of the region it is contained in) when the chip is securely positioned in the machines for centrifugation. In various embodiments, when the chip is rotated 180 degrees and securely repositioned in the centrifugation machines for centrifugation, the longitudinal axis or the line of symmetry of each of the device (or the regions within the device) still approximately passes through the axis of rotation (to allow the centrifugal force to direct the cells contained therein to the other end of the region it is contained in) of the centrifugation machines.

In various embodiments, usefully, a scaled-up (chip or plate) version of the device (i.e., microfluidic device) may be mass produced and made accessible in the form of a commercial consumable, along with a protocol/instructions on how to successfully use the consumable. Such consumables may also be further tagged for traceability purposes.

Various embodiments of the device disclosed herein may provide a design with built-in amenability to scale-up using at least two types of multiplexed devices in the form of a chip or plate.

There is also provided a method of interacting a first cell type with a second cell type (or studying interactions between a first cell type with a second cell type, or screening a sample comprising a first cell type). The method may include providing the first cell type in a first region of the device provided herein; applying an external force (e.g. rotational force or centrifugal force) to the first cell type to direct the first cell type to an interaction zone of the first region; providing the second cell type in the second region of the device provided herein; and allowing the first cell type to migrate from the first region to the interaction zone of the second region for interaction with the second cell type in the interaction zone, with or without application of an external force. Advantageously, in various embodiments, the method provides an in-vitro method for e.g., screening individual tumor sample responses to new and existing combinatorial cancer therapies involving human immune cells.

In various embodiments, the method of studying interactions of a first cell type with a second cell type, includes providing a device disclosed herein that is structurally configured to facilitate the desired interactions between the cell types. Such device, for example, may comprise at least a first region configured to hold a first cell type, a second region configured to a second cell type, and an array of microstructures disposed between the first and second regions, wherein the first region is in fluid communication with the second region, and wherein the array of microstructures is configured to selectively allow movement of the second cell type from the second region to an interaction zone that is at least partially disposed within the first region, to allow interaction of the first cell type and the second cell type in the interaction zone. Thereafter, the method may then involve seeding the first cell type in the first region of the device. The method may also comprise applying a first external force to direct the first cell type to the interaction zone. A second cell type may be seeded in the second region of the device. The seeding of the second cell type may be carried out after the first cell type has been directed to the interaction zone by the external force. After the second cell type has been seeded, the second cell type may be allowed to migrate from the second region (e.g., across the microstructures) to the interaction zone for interaction with the first cell type in the interaction zone. Such migration of the second cell type and interaction of the second cell type with the first cell type may then be monitored.

In various embodiments, the first cell type is provided in the first region of the device and/or the second cell type is provided in the second region of the device manually/by hand (e.g., by applying an external force or by passive flow) or by using an automatic handler (e.g., a bioprinter).

In various embodiments, applying a first external force to the device to, for example, load the first cell type (e.g., tumor cells, in the form of tumor fragments for example) may be performed through a first centrifugation. In various embodiments, after the first cell type is seeded into the device, by applying the external forces (e.g., rotational or centrifugal forces), the first cell type may be directed away from the first seeding port of the device and towards the first interaction zone that is at least partially located in the first region.

In various embodiments, monitoring migration of the second cell type and/or interaction of the second cell type with the first cell type comprises monitoring with an imaging device/apparatus, for example, observing the device via microscopy (e.g., confocal microscopy).

In various embodiments, the method further comprises capturing one or more images (e.g., microscopic images) comprising the array of barriers and/or the first region and/or the interaction zone or parts thereof with an image capturing device, for example a microscope enabled with video/image capturing capabilities (e.g., linked to a computer).

In various embodiments, the method further comprises tracking/monitoring cell properties/characteristics/behaviour/interaction such as cell viability, cell count, distances travelled (e.g., distances travelled by the second cell type), cytokine/chemokine activity/measurement, clustering/recruitment of cells (e.g., recruitment of the second cell type), cell morphology, cell motility and/or speed of migration or the like. For example, the cell viability of the first cell type and/or the second cell type may be tracked/monitored. As another example, cell count of the second cell type located within the array of barriers and/or located at the interaction zone may be tracked/monitored. The tracking/monitoring may be performed via computer vision, fluorescence measurements and/or immunoassays (e.g., ELISPOT). As one example, the tracking/monitoring may be performed via an automated computer vision algorithm using MATLAB that identifies key cellular interactions. In various embodiments of the method disclosed herein, if fluorescence microscopy is relied on for the generation of quantitative results, fluorescent signals can vary slightly between testing runs and can be prone to bleaching. This may have an effect on the accuracy of the automated analysis results. Therefore, in various embodiments, improved immunostaining fluorophores may be used and the exposure of the tagged cells to incident light from the microscope used may be limited.

In various embodiments, the method further comprises applying a second external force to direct cells present within the interaction zone away from the interaction zone for retrieval and analysis. This may include applying an external force of the same nature of the first external force to the device for a second time to direct cells located in the first region (e.g., the first cell type and/or recruited second cell type) away from the interaction zone and/or towards a first seeding port of the device, and/or to direct non-recruited (e.g., non-migratory) second cell type away from the second region and/or towards a third region/a third port of the device. In this step, the device may be oriented such that the device is rotated by no less than about 90 degrees and no more than about 270 degrees, no less than about 100 degrees and no more than about 260 degrees, no less than about 110 degrees and no more than about 250 degrees, no less than about 120 degrees and no more than about 240 degrees, no less than about 130 degrees and no more than about 230 degrees, no less than about 140 degrees and no more than about 220 degrees, no less than about 150 degrees and no more than about 210 degrees, no less than about 160 degrees and no more than about 200 degrees, no less than about 170 degrees and no more than about 190 degrees, or about 180 degrees from its original position/orientation, for example the device may be rotated about an axis that is substantially perpendicular to the line of symmetry of the device/first region/second region/third region or substantially parallel to (or lying in) a plane that cuts across the centre/midline of the device (e.g., including the support layer and/or seeding layer).

Accordingly, in various embodiments, the method comprises a method of centrifuging that is able to direct cells toward and away from the first interaction zone (or the tumor trap area) for the purposes of loading and retrieval respectively. That is, in various embodiments, the method disclosed herein may use centrifugal forces to position tumour masses in microfluidic devices for the purposes of trapping such cellular fractions and for the subsequent purpose of cell retrieval. The method provided herein may further involve a second centrifugation for the purposes of removing the first and second cell types (e.g., tumor cells and immune cells respectively) e.g., after a pre-determined period, for example. In various embodiments, the external forces (e.g., centrifugal forces) used in the second centrifugation may be in opposite direction to the external forces in the first centrifugation, relative to interaction zone.

In various embodiments, the method further comprises extracting cells from a first port and/or a third port of the device manually/by hand or by using an automatic handler (e.g., a bioprinter). That is, the method allows for retrieval of cells. Advantageously, various embodiments of the method disclosed herein may allow automated processing and recovery (via centrifugation and automated handler processing). In various embodiments, the method further comprises analysing the extracted cells to identify genetic biomarkers. For example, in various embodiments, the method involves recovering cell populations of interest (e.g., effective or non-effective tumor infiltrating lymphocytes cells) that can be further studied for biomarker identification.

In various embodiments, the method comprises seeding the immune cell types and subsequently, after the experiment is finished, removing those immune cell types from the same port. In various embodiments, the tumor cell types are seeded and removed from the same port, and any immune cell types that have migrated into the tumor spheroid itself will be removed along with the tumor spheroid. In various embodiments, there is a possibility of seeding the immune cells from an immune cell recovery port. This cell recovery port may in fact be located on “the same side” as the tumor seeding port in terms of its proximity but does not mean that the two cell types would be seeded from the same port. That being said, in various embodiments, there is a possibility of seeding microenvironment materials such as a tumor microenvironment cell type (e.g., fibroblasts) from the tumor seeding port (prior to seeding the tumor spheroid itself) such that they adhere to the spaces in between the artificial pillar barriers and form a more physiologically accurate environment for the tumor spheroid. In this regard, in some embodiments, the device used for the method may further comprises one or more third regions which are capable of holding microenvironment materials (e.g., tumour microenvironment cell type or material) and the method comprises seeding microenvironment materials into the one or more third regions. The arrangement of the third region may be such that the third region is in fluid communication with the first and second regions, and the array of microstructures comprises microstructures disposed between the third region and the first region. In various embodiments, the array of microstructures also comprise microstructures disposed between the third region and the second region. Accordingly, after seeding of the microenvironment materials, these materials may be sandwiched between the first region (or first cell type after it has been seeded) and the second region (or second cell type after it has been seeded) so that the second cell type has to migrate through these materials to reach the first cell type for interaction. The seeding of the microenvironment materials in the third region may be carried out prior or after the seeding of the first cell type in the first region. The seeding of the microenvironment materials in the third region may also be carried out prior to or after seeding of the second cell type in the second region. In various embodiments, the seeding of the microenvironment materials in the third region is prior to applying the first external force (e.g., centrifugal force) to direct the first cell type to the interaction zone.

An exemplary method is described below for illustrative purposes. In one example, the method may be a method (or workflow) by which tumor-immune cell interactions can be screened at high-throughput, comprising the following steps: (a) automated depositing of tumour-related cellular matter into sealed devices; (b) centrifugation of multiple devices so as to aggregate and position cellular matter; (c) automated deposition of immune cells into the devices; (d) observation of tumour-immune interactions with microscopy and automated image analysis of said interactions to identify targeted interactions; (e) centrifugation of multiple devices to recover distinct cell populations; and (f) automated removal of cell populations for further analysis.

Various embodiments of the present disclosure may provide a combination of a device and a process/method which combines a unique microfluidic design with centrifugation, resulting in a unique way to isolate and combine two distinct cell types: tumor and immune cells, and separating them with a physical barrier, or a combination of physical and biological barriers by creating an artificial TME, in a single device such that their interactions—particularly tumor-infiltrating lymphocyte behavior—can be studied in an explicit manner.

### BRIEF DESCRIPTION OF FIGURES

FIG. 1A is a schematic diagram illustrating a three-dimensional exploded view of a single compartmentalised microfluidic screening device, in accordance with various embodiments disclosed herein. FIG. 1B is a schematic drawing illustrating a top view of the compartmentalized microfluidic screening device, in accordance with various embodiments disclosed herein. FIG. 1B shows the designated compartments, device dimensions and depth ranges.

FIGS. 2A, 2B, 2C, 2D, 2E, 2F, 2G and 2H are schematic drawings of the array of barriers (or microstructures) which creates a tumor trap inside the tumor-immune cell interaction chamber of the compartmentalized microfluidic device in accordance with various embodiments disclosed herein. Different microstructure geometries and organizations as shown in FIGS. 2A to 2H can be employed depending on need. FIG. 2I is an enlarged schematic drawing of the dotted square portion shown in FIG. 2A. In FIG. 2I, the narrowest gap between structures (on the innermost row of any barrier) is 5 μm while the largest gap is 25 μm (on the outermost row) to accommodate human T-Cell size and migration patterns.

FIG. 3A is a schematic diagram illustrating a three-dimensional exploded view of another single compartmentalised microfluidic screening device, in accordance with various embodiments disclosed herein. FIG. 3B is a schematic drawing illustrating a top view of the another compartmentalized microfluidic screening device, in accordance with various embodiments disclosed herein. FIG. 3B shows the designated compartments, device dimensions and depth ranges.

FIGS. 4A, 4B and 4C are schematic drawings of the tumor immune interaction chamber in the another compartmentalized microfluidic screening device of FIGS. 3A and 3B, in accordance with various embodiments disclosed herein. FIG. 4B is an enlarged schematic drawing of the dashed and rounded square portion shown in FIG. 4A. FIG. 4C is a schematic drawing of micropillars shown in FIGS. 4A and 4B. As shown in FIGS. 4A and 4B for example, the area containing the 3D tumor spheroid aggregates is surrounded by two or more concentric rows of rectangular pillars creating a tumor trap which holds the spheroid aggregate in place. Different materials (i.e., hydrogels or cell laden matrices) are centrifuged into position and held in place by the organized rows of pillars. The pillar sizes vary in each concentric row (see FIG. 4C) and the gaps between pillars (see FIG. 4B) can also vary while still allowing effector (immune) cells to be recruited (via chemotaxis) from the outermost layer—the effector cell region—to the tumor spheroid region.

FIG. 5 is a schematic drawing illustrating an experimental workflow using a compartmentalised microfluidic screening device, in accordance with various embodiments disclosed herein.

FIG. 6 is a schematic drawing illustrating an experimental workflow using another compartmentalised microfluidic screening device, in accordance with various embodiments disclosed herein. In FIG. 6, in the first step, tumor spheroids and tumor microenvironment (TME) materials are seeded into the device via an automated pipette. The device is then centrifuged to position the tumor cells and TME materials in the tumor-immune interaction zone (Step 2). Effector (immune) cells are then added (Step 3) and the migration and interaction of the effector cells with the tumor aggregate is continuously monitored and analyzed using microscopy and computer vision algorithms which track cell events of interest (Step 4). Post-experiment, the device is centrifuged again (reversing the direction of apparent centripetal force) to pellet the tumor cells with tumor infiltrating lymphocytes (TILs) as well as the TME materials with any immune cells that were located in those regions (Step 5). Finally, the cells and TME materials are retrieved via (automated) pipette and are analyzed further downstream (e.g., sequencing).

FIG. 7 is a schematic drawing illustrating a three-dimensional exploded view of a multiplexed (6×6) chip device conforming to the standard dimensions of a microscope slide, in accordance with various embodiments disclosed herein.

FIG. 8 is a schematic drawing illustrating a three-dimensional exploded view of a multiplexed (21×10) plate device conforming to the standard dimensions of a well plate, in accordance with various embodiments disclosed herein.

FIG. 9 is a schematic drawing illustrating a three-dimensional exploded view of a multiplexed (5×5) chip device conforming to the standard dimensions of a microscope slide, in accordance with various embodiments disclosed herein.

### EXAMPLES

Example embodiments of the disclosure will be better understood and readily apparent to one of ordinary skill in the art from the following discussions and if applicable, in conjunction with the figures. It should be appreciated that other modifications related to structural and biological changes may be made without deviating from the scope of the invention. Example embodiments are not necessarily mutually exclusive as some may be combined with one or more embodiments to form new exemplary embodiments. The example embodiments should not be construed as limiting the scope of the disclosure.

The examples describe a platform suitable for use for in-vitro chemotherapeutic and immunotherapeutic combinatorial drug testing using at least two different types of cells: tumor cells and immune cells (e.g., CD8+ T cells, Natural Killer (NK) cells, etc.) with the option of including other cell types to create a complex three-dimensional microenvironment surrounding a tumor aggregate and more accurately physiologically mimic the tumor microenvironment in vivo. The platform is comprised of a physical device, an assay protocol, and an analysis procedure designed to be amenable to process automation and scale-up.

### The Physical Device

In the following examples, a microfluidic device (i.e., a compartmentalized tumor trapping microfluidic device) for observation of immune-tumor interactions is provided. In some of the following examples, additional compartmentalized layers are provided for placement of cell-laden hydrogels or artificial extra-cellular matrix material surrounding the trapped tumor to create complex three-dimensional microenvironments.

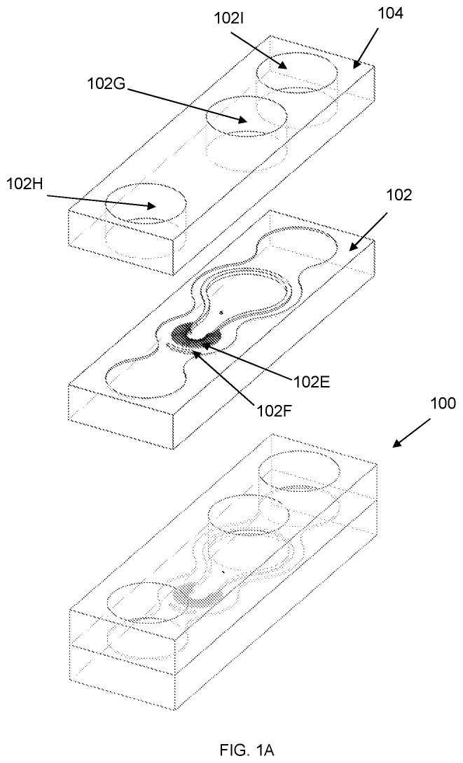

A three-dimensional illustration of an exemplary physical device is shown in FIG. 1A, and a view of the device from the top is shown in FIG. 1B to highlight the different compartments.

In FIG. 1A, the microfluidic device 100 described is a microfluidic device designed specifically with four different zones or compartments, each with a specific purpose: tumor cell seeding zone/compartment, immune cell seeding zone/compartment, controlled tumor-immune cell interaction and observation zone/compartment, and cell recovery zone/compartment.

In FIG. 1A, the single physical sealed device 100 is comprised of: 1) a support layer in the form of a fluidic layer 102 (or a cell-interaction layer) that contains an array of microstructures 102E (i.e., micro-structured architecture) necessary for partitioning the different zones/compartments as well as controlling the positioning of the different cell populations throughout the entire experimental assay, and 2) a seeding layer 104 (in the form of a cap layer or a cell seeding layer) approximately 1 mm thick that seals the fluidic layer except for the cell seeding ports (i.e., there are openings for the tumor spheroid seeding port 102G, immune cell seeding port 102H, and cell extraction port/bubble trap 102I), and encloses the tumor-immune cell interaction zone/compartment (i.e., in the fluidic layer 102) completely. The device 100 is therefore an open-well format type of device with the ports remaining open to the outside environment.

The fluidic layer 102 ranges between 25 and 200 μm in depth depending on the type of device and the region/zone/compartment area (i.e., depending on the application and type of cells being used, and the region/zone/compartment area) (see FIG. 1B). In the fluidic layer 102, a first region 102A configured to hold tumor cells (at least partially within the tumor cell seeding port zone/compartment 106) is used for the initial placement of a first cell type (i.e., tumor cells) into the device 100, in either singularized or spheroid form. The tumor cell seeding port zone/compartment 106 is large enough to accommodate the initial seeding of cells in liquid or gel media, and acts as a holding area until the cells can be directed later into a different zone/compartment. The first region 102A has a depth of from 50-200 μm. A second region 102B configured to hold immune cells (at least partially within the immune cell seeding port zone/compartment 108) serves a similar function to the first region 102A. A second cell type (i.e., immune cells) are seeded into this area in a liquid medium and allowed to move freely in the 25-50 μm in depth area shown in FIG. 1B. An interaction zone 102C (at least partially within the tumor—immune cell interaction zone/chamber 110) is a compound zone/compartment in that it is dual-tiered (each tier with different depths) and contains an array of microstructures 102E (i.e., specific microstructure architectures) for controlling the interaction between the different cell populations. The center region of the tumor—immune cell interaction zone/chamber 110 is comprised of a tumor trap, which holds the tumor cells/spheroids in place at the center of a grid or radially arranged matrix of microstructures acting as a barrier between the tumor cells and the immune cells. The fluidic layer 102 also comprises an additional obstacle 102F that reduces the likelihood of non-interacting immune cells from entering the first region 102A during centrifugation to move the interacting tumor/immune cells away from the interacting zone 102C for retrieval and analysis at the tumor cell seeding port 102G.

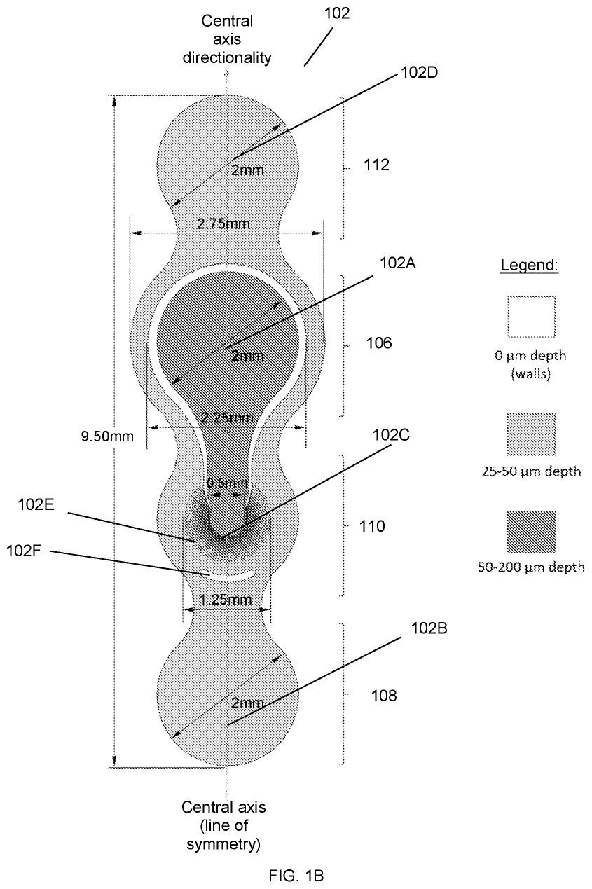

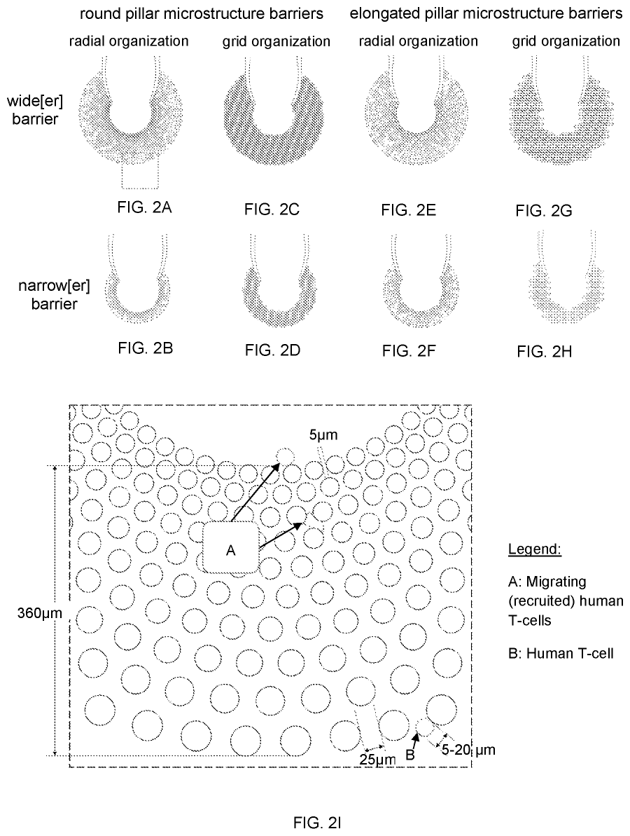

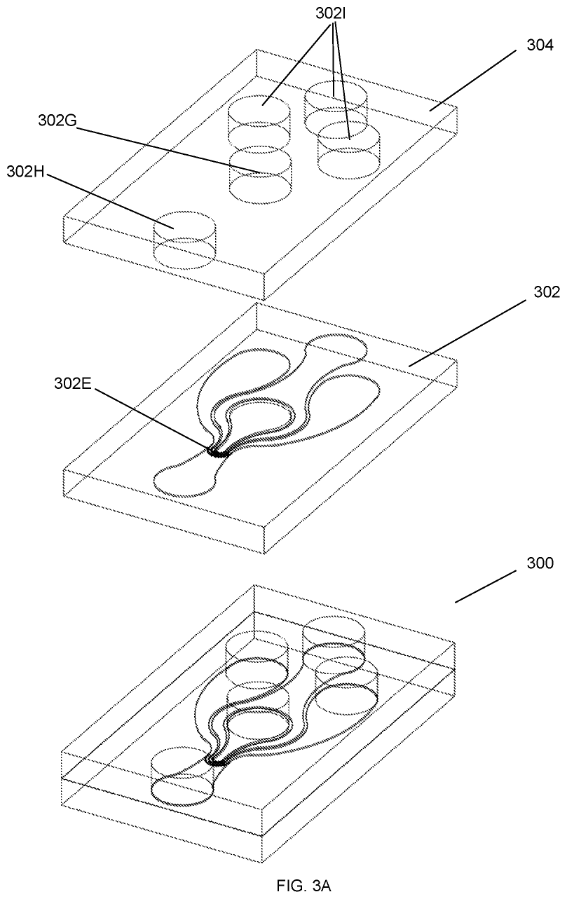

Some examples of the different types of microstructures arrays and resulting barriers are shown in FIGS. 2A to 2H. The microstructure arrays have the same height as the fluidic layer 102 and can thus range from 25 μm to 50 μm in height. The distance between each type of microstructure (i.e., barrier porosity) depends on the organization of the array. Radially-organized microstructure arrays (see FIGS. 2A, 2B, 2E and 2F) exhibit a gradient of distances between microstructures. Here, the distance between microstructures in the outermost row (farthest from the center) is equal to 25 μm whereas the distances between microstructures decrease incrementally as they come closer to the center until reaching the innermost row (closest to the center) which has a distance of 5 μm between structures. As an example, see FIG. 2I for an expanded view of the dotted section in FIG. 2A. This gradient was designed with the size distribution of a population of immune cells in mind (ranging from 5 to 20 μm). The gradient also reflects an increasing challenge posed to the immune cells in terms of the narrowness of the channel pathway that they must navigate through to move from the outside of the barrier to the inside. Grid-organized microstructure arrays (see FIGS. 2C, 2D, 2G and 2H) have a uniform distance between microstructures throughout the entire barrier, independent of their distance from the radial center. Although the type of structure may vary, the distance between structures can range from 5 to 25 μm. The challenge posed to the immune cell population in this case is consistent throughout the entire barrier.

Returning to FIG. 1B, the device 100 further comprises a third region 102D (at least partially within the cell recovery port and air bubble trap zone/compartment 112). The third region 102D and the cell recovery port and air bubble trap zone/compartment 112 of the device 100 serve two purposes: as an open exit port in contact with the surrounding atmosphere for equilibration and also so that any incident air bubbles can be pushed through the device 100. The third region 102D and the cell recovery port and air bubble trap zone/compartment 112 also serve as a cell recovery port to recover immune cell populations that did not migrate through the barrier in the tumor.

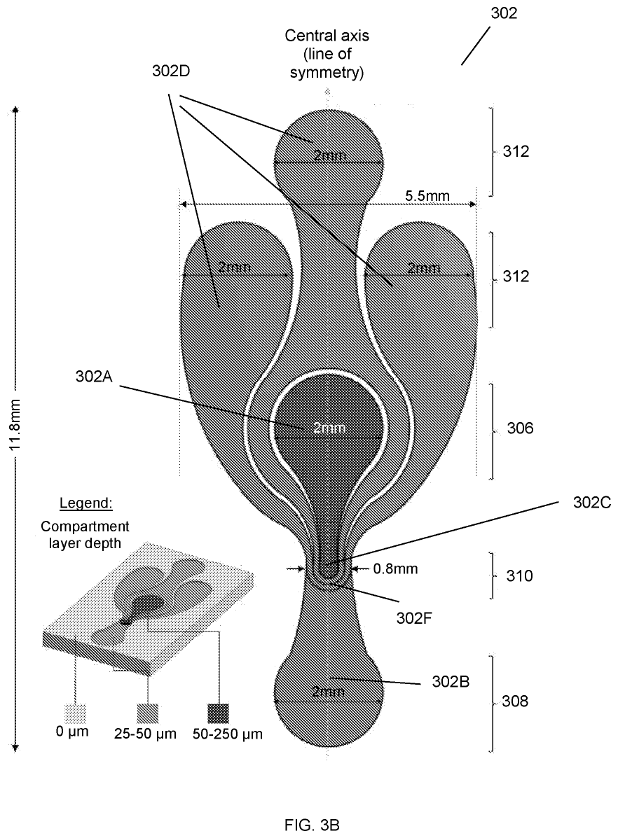

A three-dimensional illustration of another exemplary physical device 300 is shown in FIG. 3A, while a view of the device from the top is shown in FIG. 3B to highlight the different compartments.

The microfluidic device 300 is a physical device designed specifically with four or more different zones or compartments, each with a specific purpose: a tumor cell seeding port zone which can also function as a cell recovery port zone, an immune cell seeding port zone, a controlled tumor-immune cell interaction and observation zone, and a variable number of tumor microenvironment (TME) material (such as hydrogels, cellular material, or both) seeding port zones which can also function as cell recovery port zones.

Similar to the device 100 described with reference to FIG. 1A, the device 300 of FIG. 3A is comprised of: 1) a support layer in the form of a fluidic layer 302 (or cell-interaction layer) that contains an array of microstructures 302F (i.e., micro-structured architecture) necessary for partitioning the different compartments as well as controlling the positioning of the different cell populations throughout the entire experimental assay, and 2) a seeding layer 304 (or a cap layer) approximately 1 mm thick that seals the fluidic layer 302 with openings for the cell seeding ports (e.g., tumor spheroid seeding port 302G and immune cell seeding port 302H), and encloses the tumor-immune cell interaction zone/compartment completely. The device 300 is therefore an open-well format type of device with the ports remaining open to the outside environment.

Also similar to the device 100 described with reference to FIG. 1A, the fluidic layer 302 as shown in FIG. 3B comprises a first region 302A for holding tumor cells/spheroids with a depth of between 50-250 μm (that is at least partially within a tumor cell seeding port zone/compartment 306), a second region 302B for holding immune cells with a depth of between 25-50 μm (that is at least partially within an immune cell seeding port zone/compartment 308) and an interaction zone 302C (that is at least partially within a tumor-immune cell interaction zone/chamber 310). In FIG. 3B, the fluidic layer 302 further comprises at least two third regions 302D (that are at least partially within the Matrigel/fibroblast seeding port zones/compartments 312). The third regions 302D and the Matrigel/fibroblast seeding port zones/compartments 312 allow tumor microenvironment (TME) materials to be provided and to be removed. TME materials include cancer associated fibroblasts or artificial extra cellular matrices such as Matrigel.

In FIG. 3B, the center region of the tumor/immune interaction zone/chamber 310 is comprised of a tumor trap, which holds the tumor cells/spheroids in place at the center of one or more radially arranged rows of micropillars 302F.

These concentric rows of micropillars keep the tumor spheroid in place, and allow for the organized placement (via centrifugation) of layers of tumor-microenvironment materials. There are spaces between each of the micropillar structures, allowing for effector (immune) cells migrating from the outer regions of the tumor-immune interaction zone to the innermost region (where the tumor spheroid is located) to pass through.

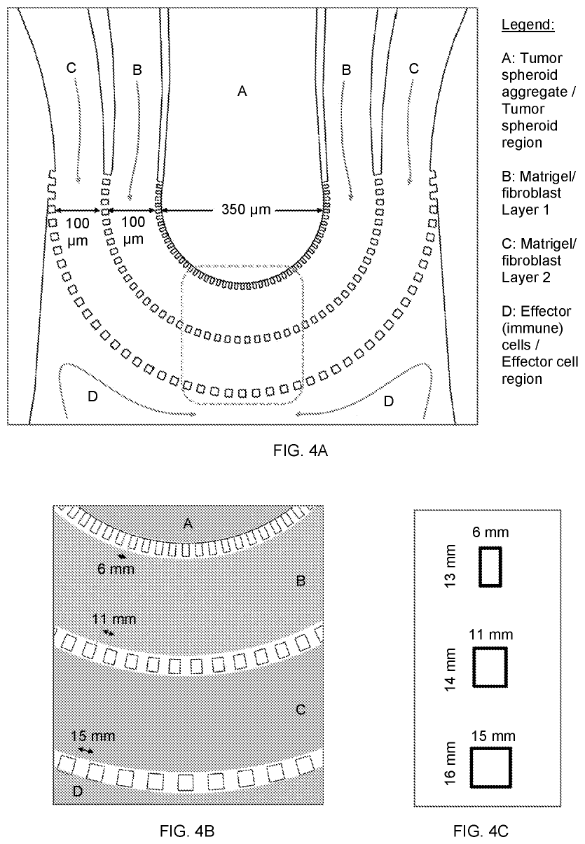

An example of the arrangement of the micropillar barriers in the tumor-immune interactions zone creating two distinct layers surrounding the tumor spheroid regions is shown in FIGS. 4A to 4C. The micropillar structures form the physical barrier around the tumor spheroid and have the same height as the fluidic layer and can thus range from 25 μm to 50 μm in height. The distance between each type of microstructure (i.e., barrier porosity) can vary. In the example shown in FIG. 4B, the distance between the micropillars in the outermost row (farthest from the center) of radially arranged micropillars is equal to 15 μm whereas the distances between micropillar structures in the rows closer to the tumor-spheroid region decrease incrementally as they come closer to the center until reaching the innermost row (closest to the center) which has a distance of approximately 6 μm between structures. This gradient was designed with the size distribution of a population of immune cells in mind (ranging from 5 to 20 μm). The gradient also reflects an increasing challenge posed to the immune cells in terms of the narrowness of the channel pathway that they must navigate through to move from the outside of the barrier to the inside. As indicated in FIGS. 4A and 4B, in between the radially organized rows of micropillars different materials can be strategically placed to form a complex three-dimensional biological barrier between the effector (immune) cells and their tumor spheroid target. The materials forming these layers can be positioned in an organized manner via centrifugation (see FIG. 4A). Together with the physical barriers, these biological barriers form the complex three-dimensional tumor microenvironment surrounding the tumor through which the effector cells must migrate in order to reach the tumor.

### The Experimental Assay Protocol (Workflow)

In the following examples, processes (involving the devices e.g., device 100 of FIG. 1A and device 300 of FIG. 3A) resulting in directed, controlled interactions between cell populations is described.

In the following first example, the protocol and workflow described was developed specifically for the device 100 described with reference to FIG. 1A (which conversely was designed for streamlined processing and scale-up) and entails a multi-step process that aims to achieve three general goals: cell seeding and localization, observation of cellular interactions, and cell recovery.

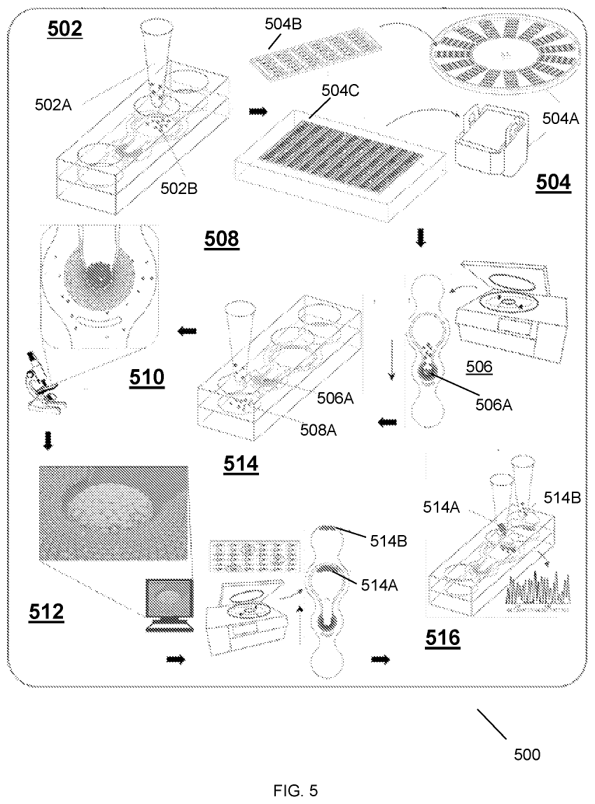

FIG. 5 provides an overview of the protocol and workflow 500 of the assay directly involving the device 100 of FIG. 1A. The entire experimental process, including obtaining patient samples and/or patient-derived cell lines, the culturing and maintenance of these samples prior to and after the experiment and the identification of biomarkers is implicit and thus not included in the figure. In the first step (FIG. 5, step 502), a population of tumor cells/spheroids 502B are seeded into the sealed device via the tumor seeding zone/compartment using an automated handler 502A (such as a bioprinter). The device 100 is then quickly placed into a special adapter 504A (FIG. 5, Step 504) for a multiplexed chip 504B (e.g., 36 plex chip) or plate 504C (e.g., 210-plex plate) (i.e., comprising a plurality of the device 100 described with reference to FIG. 1A), and then centrifuged (FIG. 5, Step 506). As the device 100 is symmetric along a central axis, it can be arranged with this axis pointing radially towards or away from the center of the centrifuge (central axis and directionality of central axis shown in FIG. 1B). In FIG. 5, Step 506, the device 100 is oriented with its central axis pointing towards the center of the centrifuge and the resulting apparent centrifugal force thus acts in the opposite direction. This apparent centrifugal force (shown by the downward arrow) directs the recently seeded tumor cells/spheroids 506A towards the tumor-immune cell interaction zone/chamber and into the trap area where they are trapped by the array of microstructures, which act as a barrier in this compartment, hold the tumor cells in a centralized position. After centrifugation, the device 100 is removed and the immune cell population 508A is added via a port 102B in the immune cell seeding port zone/compartment (FIG. 5, Step 508) of the sealed device, again using an automated handler. The device, with both cell populations seeded and in position, is now primed for observation via microscopy (FIG. 5, Step 510). Immune-tumor interactions are observed throughout the duration of the experiment and immune cells that are successfully recruited to the tumor mass by migrating through the artificial barrier separating the two cell populations are separated from the cells that are not recruited. Immune cells located inside the tumor trap region can infiltrate the tumor mass, potentially resulting in tumor cell death. In FIG. 5, Step 512, the cells located inside the tumor mass (successful infiltration) and in the tumor trap region as well as other immune cells located at various positions inside and along the artificial barrier are tracked, monitored and recorded using an automated computer vision analysis procedure which includes using a computer vision algorithm to detect and identify targeted interactions. In FIG. 5, Step 514, the device 100 is centrifuged once more but with its central axis pointing away from the center of the centrifuge (i.e., in the opposite direction as Step 506) to recover cell populations in devices which exhibited interesting cellular interactions. The apparent centrifugal force (shown by the upwards arrow) now directs the cells which are located in the tumor trapping region (both tumor and recruited immune cells 514A) back into the tumor cell seeding port zone/compartment. Any non-recruited (non-migratory) immune cells 514B will be directed to the opposite end of the device 100 towards the cell recovery port zone/compartment. In the last step (FIG. 5, Step 516), the separated, phenotypically different cell populations are recovered with an automated handler and analyzed to identify genetic biomarkers that can be used to determine responsiveness to treatment.

In the following second example, the protocol and workflow described was developed specifically for the device 300 described with reference to FIG. 3A (which conversely was designed for streamlined processing and scale-up) and entails a multi-step process that aims to achieve three general goals: 1) the creation of a complex three dimensional tumor microenvironment (involving cell seeding and organization of TME materials in stratified layers, 2) observation of cellular interactions, and 3) cell recovery.

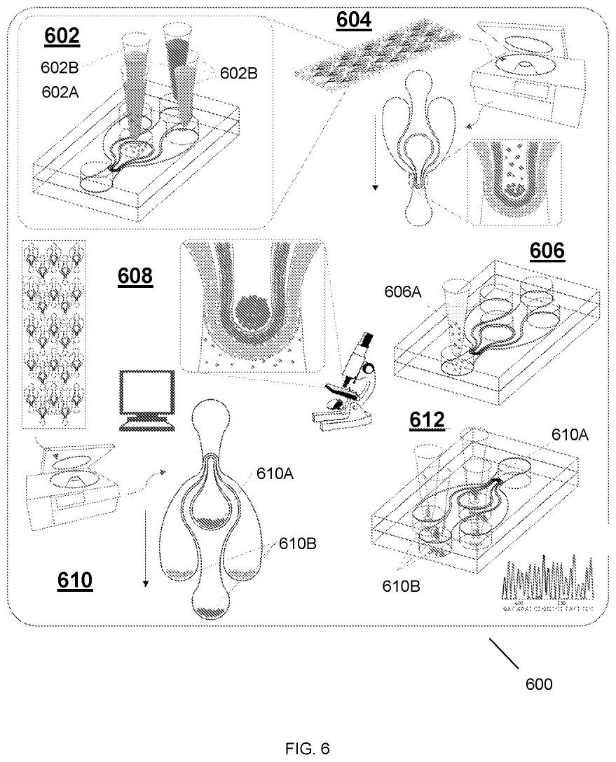

FIG. 6 provides an overview of the protocol and workflow 600 of the assay directly involving the device 300 of FIG. 3A. The entire experimental process, including obtaining patient samples and/or patient-derived cell lines, the culturing and maintenance of these samples prior to and after the experiment and the identification of biomarkers is implicit and thus not included in the figure. In the first step (FIG. 6A, Step 602), a population of tumor cells/spheroids 602A as well as tumor microenvironment materials 602B such as cancer associated fibroblasts or artificial extra cellular matrices such as Matrigel, are seeded into the device via different seeding ports (ports 302G and 302I) using an automated handler (such as a bioprinter or a pipette). A multiplexed chip or plate (i.e., comprising a plurality of the device 300 described with reference to FIG. 3A) is prepared, and the device 300 is then centrifuged (FIG. 6A, Step 604). At Step 604, if a multiplexed chip is used, to fit the multiplexed chip into centrifuges, the chip is placed inside an adaptor, for example, a plastic falcon tube. If a multiplexed plate is used at step 604, the multiplexed plate with its larger dimensions would require a different type of specialized adaptor (e.g., an adaptor that can accommodate the larger dimensions of the multiplexed plate). As the device 300 is symmetric along a central axis, it can be arranged with this axis pointing radially towards or away from the center of the centrifuge (central axis and directionality of central axis shown in FIG. 3B). The device 300 is oriented with its central axis pointing towards the center of the centrifuge and the resulting apparent centrifugal force (shown by the downward pointing arrow) thus acts in the opposite direction. The apparent centrifugal force directs the recently seeded tumor cells and TME materials towards the tumor-immune cell interaction zone/chamber where they are trapped by the array of microstructures. These micropillar microstructures serve as a barrier in this compartment (i.e., the tumor-immune cell interaction zone/chamber) and hold the tumor cells in a centralized position while also creating stratified layers of materials to which create the complex three-dimensional TME (i.e., a biological barrier). After centrifugation, the device 300 is removed and the immune cell (effector cell) population 606A is added via the port 302H in the immune cell seeding port zone/compartment (FIG. 6A, Step 606), again using an automated handler. The device 300, with both the tumor and immune cell populations seeded and in position, is now primed for observation via microscopy (FIG. 6A, Step 608). Immune-tumor interactions are observed throughout the duration of the experiment and immune cells are allowed to migrate through the different barriers (both physical and biological). The immune cells are thus separated into different populations based on the extent to which they can migrate through barriers. Immune cells located inside the tumor trap region can infiltrate the tumor mass, potentially resulting in tumor cell death. The cells located inside (successful infiltration) and around the tumor mass as well as other immune cells located at various positions within the tumor-immune interaction zone are tracked, monitored and recorded using an automated computer vision analysis procedure, which includes an automated machine learning computer vision algorithm that identifies devices of interest with tumor infiltrating lymphocytes (TILs). In FIG. 6A, Step 610, the device 300 is centrifuged once more but with its central axis pointing away from the center of the centrifuge. The apparent centrifugal force (shown by downward pointing arrow) now directs the cells which are located in the tumor trapping region (both tumor and recruited immune cells 610A, such as infiltrating lymphocytes (TIL) that penetrated into the tumor area) back into the tumor cell seeding zone/compartment. The apparent centrifugal force also directs the TME materials 610B (e.g., cancer associated fibroblasts, Matrigel, etc.) that were surrounding the tumor spheroid back into their seeding port regions along with any immune cells that might have been positioned within those barriers at the end of the experiment. In the last step (FIG. 6A, Step 612), the separated, phenotypically different cell populations are recovered with an automated handler and can be analyzed further to identify genetic biomarkers that can be used to determine responsiveness to treatment.

### The Automated Analysis Procedure

In the examples described above e.g., with reference to FIGS. 5 and 6, a computer vision algorithm to identify, track and monitor cellular interactions is described.

In the examples described above, microscopic images captured at a frequency of 2 to 4 images per hour are recorded throughout the duration of the experiment and analyzed using a computer vision algorithm written specifically to identify, track and flag cellular interactions of interest, namely: tumor cell viability, immune cell viability and immune cell count (within the barrier and inside the tumor trap area).

In the examples described above, the algorithm is designed to simplify and streamline the analysis process (e.g., see FIG. 5, Step 512, and FIG. 6, Step 608) thereby expediting the rate at which devices of interest in an array of devices can be identified (e.g., see FIG. 5, Step 514, and FIG. 6, Step 608) and methodically recovered (e.g., see FIG. 5, Step 516, and FIG. 6, Step 612).

### Platform Flexibility

In the examples described e.g., with reference to FIGS. 5 and 6, a multiplexed chip or plate device has been described.

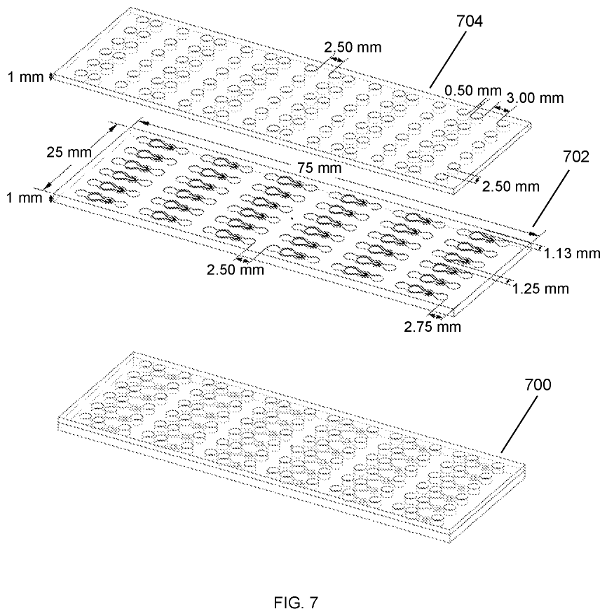

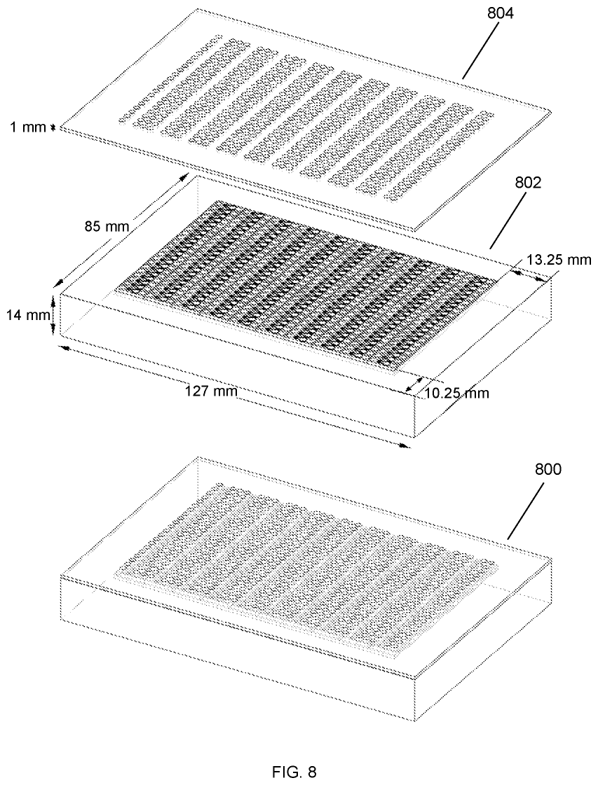

Two different exemplary multiplexed devices (based on the device 100 described with reference to FIG. 1A) that conform to industry standard dimensions (the size of a standard microscope slide or a well plate) are presented in FIGS. 7 and 8, respectively. In FIG. 7, a multiplexed (6×6) chip device 700 conforming to the standard dimensions of a microscope slide is shown. The chip device 700 comprises a fluidic layer 702, which contains numerous micro-structured architecture, and a cap layer 704, which seals the fluidic layer 702 except for the numerous cell seeding ports. In FIG. 8, a multiplexed (21×10) plate device 800 conforming to the standard dimensions of a well plate is shown. The plate device 800 similarly comprises a fluidic layer 802, which contains numerous micro-structured architecture, and a cap layer 804, which seals the fluidic layer 802 except for the numerous cell seeding ports.

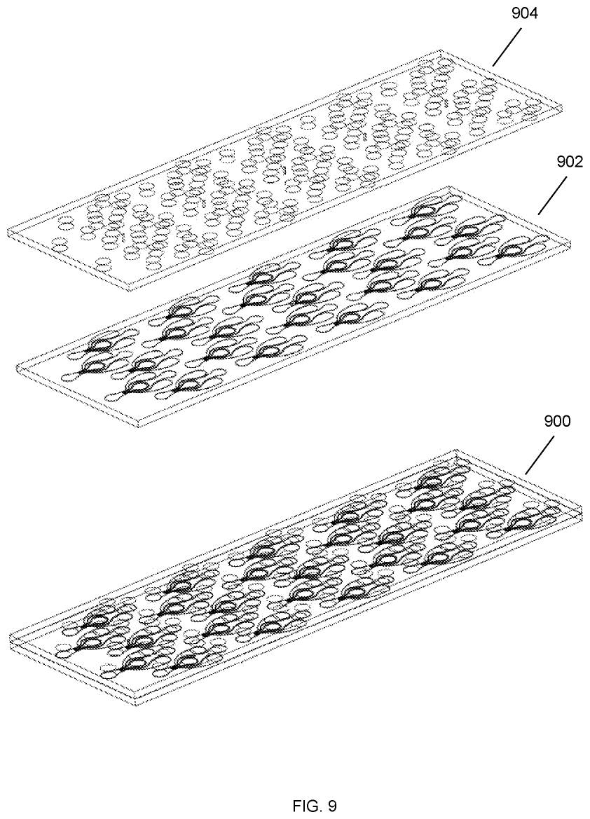

Another exemplary multiplexed device (based on the device 300 described with reference to FIG. 3A) that conform to industry standard dimensions (the size of a standard microscope slide) is presented in FIG. 9. In FIG. 9, a multiplexed (5×5) chip device 900 conforming to the standard dimensions of a microscope slide is shown. Similar to the chip device 700 described with reference to FIG. 9, the chip device 900 comprises a fluidic layer 902, which contains numerous micro-structured architecture, and a cap layer 904, which seals the fluidic layer 902 except for the numerous cell seeding ports.

In FIGS. 7, 8 and 9, the devices shown have been designed such that the orientation of the individual microfluidic compartments maintain their alignment and can still be centrifuged with nearly identical centrifugal force. The devices can be used in existing imaging solutions present in the laboratory ecosystem (e.g., confocal microscopes, plate readers, etc.) and exhibit sufficient spacing between individual cell seeding/recovery ports such that automated handlers (e.g., bioprinters) can still be used.

### APPLICATIONS

Advantageously, various embodiments of the method/process (including the use of the device) disclosed herein provide an in vitro assay that can be scaled-up easily and used for high-throughput screening that would allow pharmaceutical companies and clinical researchers for example to study patient responses to specific therapies without posing danger to the patient, as well as provide an early-stage screening for healthcare providers for example to determine which patients may or may not respond well to a particular combination therapy.

Various embodiments of the present disclosure provide an OncoMiMIC (Onco-Multi-Metric Immuno-Combinatorial) testing platform. In various embodiments, the OncoMiMIC testing platform is advantageously adapted for forming a complex 3D Tumor Microenvironments (TME). In various embodiments, such platforms may be referred to as an OncoMiMIC-CTM (Onco-Multi-Metric Immuno-Combinatorial Complex Microenvironment) testing platform.

Various embodiments of the present disclosure provide a testing platform that comprises a class of microfluidic chips that are inexpensive. In various embodiments of the present disclosure, the comprehensive in-vitro testing platforms can screen individual tumor sample responses to new and existing combinatorial cancer therapies, thereby potentially reducing the cost of identifying and testing target drugs in the development and discovery stage.

In various embodiments of the present disclosure, the OncoMiMIC-CTM testing platform advantageously allows for an end user to place, in a precise and well-organized manner, one or several surrounding layers of tissues or artificial extra-cellular matrix (ECM) material around a tumor sample, thereby creating a more physiologically relevant, complex, three-dimensional microenvironment for the tumor. Then, in various embodiments, similar to the way in which the OncoMiMIC platform functions, the OncoMiMIC-CTM platform allows for the interaction of effector cells (immune cells) to interact with this complex tumor structure.

In various embodiments of the present disclosure, the testing platforms provide platform flexibility in that the platforms are amenable to automation and scale-up for an industry requiring high-throughput and big data.

In various embodiments of the testing platform disclosed herein, the platform provides an easily scalable design, which allows the platform to be highly amenable to automation. It has been recognized that a single screening test can take place in an individual device (e.g., see FIGS. 1A and 3A), but the screening of tumor-immune interactions requires a very high number of repeatable experiments to generate statistically relevant data. Furthermore, there are often many combination therapies and/or drug candidates which require testing. Therefore, various embodiments of the testing platform disclosed herein advantageously provide an ideal platform (e.g., the multiplexed devices) that would be able to conduct screening experiments quickly with the same experimental conditions and with minimal human intervention.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A microfluidic device comprising
a first region configured to hold tumor cells;
a second region configured to hold immune cells; and
an array of microstructures disposed between the first and second regions,
wherein the first region is in fluid communication with the second region,
and wherein the array of microstructures is configured to selectively allow movement of immune cells, from the second region to an interaction zone that is at least partially disposed within the first region, for interaction with tumor cells in the interaction zone.

2. The device as claimed in claim 1, wherein the first and second regions are symmetrical about a same line of symmetry.

3. The device as claimed in claim 1, further comprising one or more third regions, the third region being in fluid communication with the first and second region, wherein the array of microstructures comprises microstructures disposed between the third region and the first region.

4. The device as claimed in claim 3, wherein the array of microstructures comprises microstructures disposed between the third region and the second region.

5. The device as claimed in claim 3, wherein the third region substantially surrounds the first region.

6. The device as claimed in claim 3, wherein the first, second and third regions are symmetrical about a same line of symmetry.

7. The device as claimed in claim 1 wherein the array of microstructures comprises microstructures organised in a radial manner or a grid-like manner.

8. The device as claimed in claim 1 wherein the array of microstructures comprises microstructures organised as substantially concentric rows of microstructures.

9. The device as claimed in claim 8, wherein the distance between the microstructures in the row closest to the first region is smaller than the distance between the microstructures in the row furthest from the first region.

10. The device as claimed in claim 8, wherein the size of the microstructures in the row closest to the first region is smaller than the size of the microstructures in the row furthest from the first region.

11. The device as claimed in claim 1, wherein each of said regions comprises a shape defined by tapering of a bigger area to a smaller area.

12. The device as claimed in claim 1, further comprising ports corresponding to each of said regions for providing access to each of the regions.

13. The device as claimed in claim 12, wherein the device comprises a seeding layer and a support layer, wherein the ports are disposed on the seeding layer and the corresponding regions are disposed on the support layer.

14. The device as claimed in claim 1, wherein the first region has a larger depth than the second region.

15. The device as claimed in claim 1, wherein the second region has substantially the same depth as the third region.

16. A chip comprising a plurality of the device of claim 1.

17. A method of studying interactions of a first cell type with a second cell type, the method comprising:
providing a device comprising a first region configured to hold a first cell type; a second region configured to a second cell type; and an array of microstructures disposed between the first and second regions, wherein the first region is in fluid communication with the second region, and wherein the array of microstructures is configured to selectively allow movement of the second cell type from the second region to an interaction zone that is at least partially disposed within the first region, to allow interaction of the first cell type and the second cell type in the interaction zone;
seeding the first cell type in the first region of the device;
applying a first external force to direct the first cell type to the interaction zone;
seeding the second cell type in the second region of the device;
allowing the second cell type to migrate from the second region to the interaction zone for interaction with the first cell type in the interaction zone; and
monitoring migration of the second cell type and interaction of the second cell type with the first cell type.

18. The method of claim 17, wherein the method further comprises, subsequent to the monitoring step, applying a second external force to direct cells present within the interaction zone away from the interaction zone for retrieval and analysis.

19. The method of claim 17, wherein the monitoring step comprises monitoring the migration and interaction of the cells with an image capturing apparatus.

20. The method of claim 17, wherein the device further comprises one or more third regions, and the method further comprises, prior to the step of applying the first external force, seeding microenvironment materials into the one or more third regions,
wherein the third region is in fluid communication with the first and second regions, and wherein the array of microstructures comprises microstructures disposed between the third region and the first region.
